(19) United States
(12) Patent Application Publication
(10) Pub. No.: US 2019/0254764 A1
(43) Pub. Date: Aug. 22, 2019

(54) SYSTEMS FOR REMOVING FALLOPIAN TUBE OBSTRUCTIONS

(71) Applicant: Daniel Rostein, Oak Brook, IL (US)

(72) Inventor: Daniel Rostein, Oak Brook, IL (US)

(21) Appl. No.: 15/900,923

(22) Filed: Feb. 21, 2018

(51) Int. Cl. A61B 34/00; A61B 17/22; A61B 1/313; A61B 17/42; A61B 17/00

(52) U.S. Cl. CPC A61B 34/73; A61B 17/22031; A61B 1/3132; A61B 2034/2055; A61B 17/00234; A61B 2034/731; A61B 2017/00876; A61B 17/42

(57) ABSTRACT

The present disclosure provides devices and methods of removing a fallopian tube obstruction by placing a pair of magnets on opposite sides of a fallopian tube obstruction. The natural attraction of the magnets to each other provide pressure on the obstruction. The created pressure force can be used to capture the obstruction for removal.

### BACKGROUND OF THE INVENTION

The present subject matter relates generally to systems for removing obstructions, particularly fallopian tube obstructions.

The obstruction of any of numerous lumens in the human body is a common ailment requiring a variety of medical surgical treatments to correct the condition. For example, obstructions in the fallopian tubes of a female's reproductive system or in the bile duct of the gastrointestinal system are but two of such conditions. A major cause of infertility in woman is distal fallopian tube disease in which the inner surface or the outside of the fallopian tube develops lesions which partially or completely obstruct the lumen through the fallopian tube.

Specifically, fallopian tubes are an important part of the female reproductive system, with the delivery of eggs and important role in the uptake of transporting the fertilized egg to the uterus cavity. Fallopian tube blockages and corresponding diseases are a major culprit of female infertility, specifically accounting for about 50% of female infertility. Moreover, this disease has no age or race restrictions. For women of childbearing age have a tubal occlusion, treatment is very difficult.

Fallopian tube obstruction represents a heterogeneous group of underlying pathology, preliminary intrinsic occlusion or extrinsic compression from estrogen-sensitive disorders, such as endometriosis, adenomyosis, endosalpingiosis, and myomata. Fallopian tube obstruction is frequently diagnosed by hysterosalpingography besides laparoscopy.

A typical treatment for fallopian tube obstruction involves a surgical procedure either through a laparoscope or a more extensive laparotomy in order to excise the lesions. The procedure involves accessing the distal end of the fallopian tube and inserting forceps in the distal end of the tube. The forceps then is expanded and, in the expanded condition, is withdrawn through the tube. The procedure is repeated a number of times with the forceps being rotated each time to a slightly different angular position in an effort to define a uniform widened opening. The procedure requires a high degree of care and is time consuming. Often it results in bleeding and the development of scar sue. Notwithstanding the difficulties in the procedure, it has been widely practiced for many years. It is among the general objects of the invention to provide a novel catheter and new and improved techniques for treating an obstructed lumen, such as, for example only, a fallopian tube, bile duct or the like.

Typically, after laparoscopic removal of endometriotic lesions, Danazol or LHRH agonist (for a period of six months) is prescribed to the women treated. However, women treated with Danazol might experience gastrointestinal and hepatic disorders as well as severe androgenic side effects.

Yet another treatment method includes micro-surgery can be used as treatment of tubal infertility by removing the fallopian tube obstruction. Although the short-term postoperative recanalization rate can be up to about 90%, however, after a year the tubal occlusion rate can be as high as 61.25% 71.05%, while the incidence of ectopic pregnancy also increases significantly. This is due to the surgery to remove the obstruction in the fallopian tube actually causing a new trauma, postoperative adhesions, and scar tubal obstruction.

In addition, selective salpingography and recanalization can be used as treatment and includes the use of a coaxial catheter system with a wire guide for transvaginal recanalization of stenosed fallopian tubes, also carried out through the cervical canal. This procedure usually is performed in conjunction with diagnostic radiography prior to and following recanalization. Inasmuch as the stenosis usually is a result of inflammatory changes and is one major cause of female infertility, and since surgical transabdominal recanalization of the fallopian tubes has been shown to meet with only limited success, development of new instrumentation enabling use of non-invasive procedures is of major importance.

Accordingly, there is a need for a new non-invasive technology that can relieve the fallopian tube obstruction to reduce infertility without causing damage to the fallopian tubes.

### BRIEF SUMMARY OF THE INVENTION

The present disclosure provides a device and method for removing fallopian tube obstructions. Various examples of the device and methods are provided herein.

The present disclosure provides devices and methods of removing a fallopian tube obstruction by placing a pair of magnets on opposite sides of a fallopian tube obstruction. The natural attraction of the magnets to each other provide pressure on the obstruction. The created pressure force can be used to capture the obstruction for removal. Alternatively, the created pressure force can be an adequate amount to essentially crush or disintegrate the obstruction into particles that no long obstruct the fallopian tubes. In an example, the method includes placing magnets on either side of a tubal occlusion, wherein the magnets essentially create a fistula, the removal of which reopens the tube.

An advantage of the present system is providing a technique that can be performed with minimal expertise, compared to micro surgery with tubal anastomosis. For example, there are few surgeons that can perform a tubal reversal utilizing microsurgery. As a result, microsurgery is inconvenient and expensive. In contrast, the present system can be used by almost any gynecologist.

An advantage of the present system is providing infertility treatment for patients with previous tubal ligation and distal tubal occlusion.

An advantage of the present system is providing treatment for patients with fallopian tube obstructions with using minimal invasive surgery instead of the conventional laparotomies. As a result of utilizing minimal invasive surgery, less trauma and bleeding occurs. In addition, minimally invasive surgery is less painful, lower cost, requires less pain medicine in recovery, and results in a faster recovery.

An advantage of the present system is providing a treatment for infertility without the use of invasive surgery.

A further advantage of the present system is providing a treatment for infertility without the use of pharmaceuticals that are associated with unwanted side effects.

Yet another advantage of the present system is providing a treatment method that can be completed in less than an hour, compared to conventional techniques that require two to four hours for completion.

Another advantage of the present system is providing a system for removing fallopian tube obstructions without causing further damage and increased scar tissue.

Another advantage of the present system is improved success as compared to tubal reversals or in vitro fertilization that is successful in only 50% of the cases with major surgeries that are expensive.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a schematic of an example of the system disclosed herein.

FIG. 2 is a schematic of an example of the system disclosed herein including a positioning magnet.

FIG. 3 is a schematic of an example of the system disclosed herein wherein the obstruction is removed with the implanted magnets.

### DETAILED DESCRIPTION OF THE INVENTION

The present system utilizes the attractive force between two magnets to remove fallopian tube obstructions. Specifically, the present system includes a removeable, implantable device including two permanent magnets, each attached to a wire, wherein the wire can be used via a cannula to implant and remove the implanted magnets.

In an example, a first magnet is implanted on a first side of the obstruction within the fallopian tube and a second magnet is implanted on a second side of the obstruction within the fallopian tube. The magnets can be attached to wires that can be used to aid in implanting and removing the magnets, for example, via a cannula. As the magnets are naturally attracted to each other, the magnets move towards each other and contact the obstruction. In an example, once the obstruction is captured by the magnets, the magnets with the obstruction, can be removed from the fallopian tube via the cannula. The magnets essentially form a fistula, forming a pressure fit around the obstruction. The removable obstruction can be created in two to eight days, five to seven days, or six to seven days. After the fistula is formed, the magnets encapsulating the obstruction is removed.

In another example, as the magnets continue to move towards each other, the obstruction is destroyed by essentially crushing the obstruction into small pieces that no longer obstruct the fallopian tube. After the obstruction is decreased or destroyed, the magnets can be removed.

In an example, the system is positioned using a laparoscopy, robotic assisted laparoscopy, or any other method of minimal invasive surgery as well as laparotomy. In an example, the surgery method can include 0.3-0.5 centimeter incisions for insertion. For example, a laparoscopy utilizing four ports of entrance (e.g., for a 5 millimeter trocar). One port can be used for the insertion of a camera and three ports for the present device. A positioning cannula can be used to feed a positioning instrument that can be used to aid in positioning the two magnets around the obstruction, wherein the positioning instrument can be removed after placement of the magnets. Additional cannulas can be used to feed the capturing magnets attached to wires, wherein the capturing magnets can be removed after the suitable amount of time after which the obstruction is captured by the magnets. In an example, the cannulas are 3 millimeter cannulas inserted to allow the interruption of the proximal and distal magnets to repair the fallopian tube.

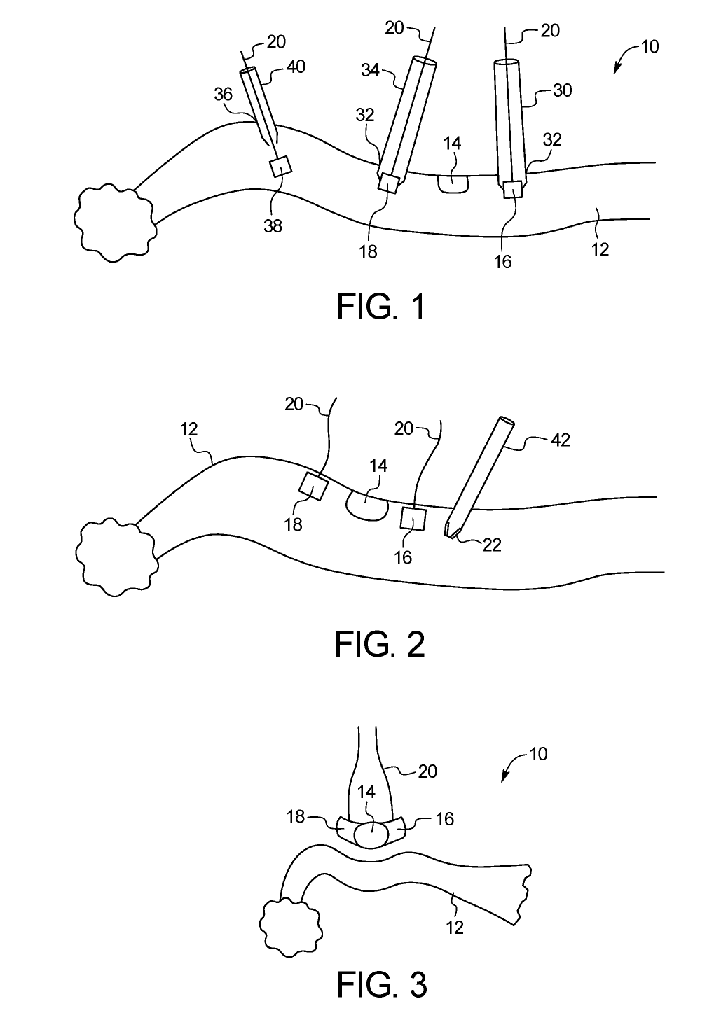

In an example, as shown in FIG. 1, the system 10 can include a first port 32 that can be used to insert a cannula 30 into the fallopian tube 12 with a first proximal magnet 16 for placement on a first side of the obstruction 14. A second cannula 34 can be inserted into the same port or a different port 32 that is located in the midline lower abdomen, wherein the second cannula 34 includes a second distal magnet 18 for placement of the second magnet 18 on the opposite side fallopian tube obstruction 14. For example, superiorly located 4 centimeters lateral from the umbilicus bilaterally, the second cannula with the second distal magnet can be introduced through the fimbria of the fallopian tube. The magnets can be placed into the fallopian tube via a 3 millimeter incision in each placement within the fallopian tube.

Once the magnets are placed within the fallopian tube, the cannula(s) can be removed and the incision closed. In an example, the magnets remain attached to a wire 20 even after the cannulas are removed, as shown in FIG. 2. The wires 20 can be used for recovering the mated magnets with the obstruction. Specifically, the magnets attract to each other and left in place for approximately one week, after which it can be removed with the obstruction, as shown in FIG. 3.

In an example, a third port 36 can be placed in the umbilical area via a third cannula 40 for the introduction of a camera 38 that enables visualization of the remaining ports. Further, the system 10 can include a positioning magnet 22 attached to a distal end of a positioning cannula 42 in order to lift and lower the placed magnets into the correct place around the obstruction, as shown in FIG. 2. For example, a cannula can be used to feed a positioning magnet that can be used to interact with the implanted magnets (e.g., capturing magnets) to move the magnets into place around the obstruction using the magnet's attraction to the magnetic cannula.

The implanted magnets can be in a variety of shapes. At least the first magnet and/or the second magnet can include an inner cavity, wherein upon mating, the magnets can house the obstruction within the cavity. In yet another example, the first magnet and the second magnet can include a passageway, wherein upon mating, the magnets can form a passageway around or through the obstruction. In an example, the first magnet can include bullet-shaped end and the second magnet can include a bullet-shaped recess, or vice versa.

Any suitable magnets can be used. For example, alnico, iron alloys, which in addition to iron can include aluminum, nickel, cobalt, copper, and/or titanium. Alnico alloys are magnetized to produce strong magnets. Each of the magnets can have a dipole axis (north pole to south pole axis). The magnets are arranged on each side of the obstruction such that the segments of the respective dipole axes are attracted to each other. For example, the obstruction is between the north dipole of the first magnet and the south dipole of the second magnet.

The magnet can be a rare earth magnet such as a neodymium magnet or a samarium-cobalt magnet. A neodymium magnet (also known as NdFeB, NIB or Neo magnet) is a permanent magnet made from an alloy of neodymium, iron and boron to the Nd₂Fe₄B tetragonal crystalline structure.

Each magnet end can be contained within or partially within a magnet housing. The magnetic housing can be any suitable material that is biologically safe, such as any suitable polymeric material. For example, the permanent magnet can be coated with a non-absorbable biocompatible substance such as nylon, polyester, or polypropylene.

A wire can be attached to the magnet and/or the magnet housing that may be used to position and remove the magnets from the fallopian tubes. The wire can be made of any suitable biologically inert material including metal, plastic, biodegradable materials, among others.

The magnet housing can also include an imaging device, wherein upon placement within the fallopian tube, the magnets can be used to image the obstruction for further assessment for treatment and/or evaluation. In addition, the imaging can be used to determine at what point the obstruction is captured or destroyed in order to indicate when the magnets can be removed.

The magnet housing can also include a medicament dispensing mechanism in order to release a substance to aid in the destruction of the obstruction. The medicament can include antibiotics, pain management medicaments, among other agents. The medicament agents can be applied or coated to one or more surfaces of the magnet housing. The agents can be included in the polymers during fabrication of the magnet housing. Alternatively, or in addition to, the agents can be included within an absorbable component attached to the magnets to provide controlled or profiled release of the substances during treatment, for example while the magnets are in contact with the obstruction.

It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

We claim:

1. A device for unblocking a fallopian tube obstruction comprising:
a first magnet connected to a first end of a first wire;
a second magnet connected to a second end of a second wire;
at least one cannula for inserting the first magnet and second magnet into a fallopian tube, wherein a second end of the first wire and a second end of the second wire extend out of the cannula when the first magnet and the second magnet are inserted into the fallopian tube.

2. The device of claim 1, further comprising a camera for visualizing the placement of the first magnet and the second magnet within the fallopian tube.

3. The device of claim 1, further comprising a positioning magnet, wherein the positioning magnet is used to attract the first magnet and the second magnet

4. The device of claim 1, wherein the first magnet and the second magnet are neodymium magnets.

5. A method of removing a fallopian tube obstruction, the method comprising:
inserting a first magnet on a first side of a fallopian tube obstruction;
inserting a second magnet on a second side of the fallopian tube obstruction;
forming a captured obstruction using the magnetic attraction of the magnets, wherein the fallopian tube obstruction is pressure fit between the first magnet and the second magnet; and
removing the captured obstruction from the fallopian tube.

6. The method of claim 5, wherein the first magnet and the second magnet are inserted through a first cannula.

7. The method of claim 5, wherein the first magnet and the second magnet are attached to a wire, wherein the wire is used to pull the captured obstruction out of the fallopian tube via a cannula.

8. The method of claim 5, further comprising inserting a positioning magnet used to move the first magnet and the second magnet into a starting position, wherein the starting position includes the first magnet on the first side of the fallopian tube obstruction, and the second magnet is on the second side of the fallopian tube obstruction.

9. The device of claim 5, wherein the first magnet and the second magnet are neodymium magnets.
